(19) United States
(12) Patent Application Publication
(10) Pub. No.: US 2003/0232380 A1
(43) Pub. Date: Dec. 18, 2003

(54) Probe array substrate for detecting target substrance

(76) Inventor: Ayako Maruyama, Kanagawa (JP)

Correspondence Address: MORGAN & FINNEGAN, L.L.P., 345 PARK AVENUE, NEW YORK, NY, 10154, US

(21) Appl. No.: 10/463,250

(22) Filed: Jun. 17, 2003

(30) Foreign Application Priority Data
Jun. 18, 2002 (JP) 2002/177,283

(51) Int. Cl. C12Q 1/68; C07H 21/04; C12M 1/34

(52) U.S. Cl. 435/6; 435/287.2; 536/24.3

(57) ABSTRACT

A probe array substrate is provided with a discrimination element using a material that changes color and/or shape due to the temperature change or contact with water or the like, whereby whether or not the probe array substrate has been already subjected to detection process of a target material can be easily determined.

### BACKGROUND OF THE INVENTION

1. Field of the Invention

The present invention relates to a method for knowing whether a probe array substrate has already been used for detection of a target substance or not. The present invention also relates to a substrate with immobilized probes provided with a discrimination element usable for such determination. More specifically, the present invention relates to a detection method capable of easily determining on the spot whether or not a substrate with immobilized probes has already been subjected to a target-detection procedure.

2. Related Background Art

As a quick and accurate method for determining nucleic acid sequence, detecting a target nucleic acid in a sample, or identifying bacterial species, a method has been proposed in which probes, i.e., a substance that specifically binds to a target nucleic acid of a specific base sequence, are immobilized in an array pattern on a solid phase to form a probe array substrate, and specific binding abilities of plural probes are evaluated at a time.

In recent years, research and development on detection and quantitative determination of a target material using the solid phase probe array has been energetically conducted. For example, U.S. Pat. No. 5,688,642 describes a solid phase oligonucleotide array prepared using photolithography, and PCT publication WO 95/25116 and U.S. Pat. No. 5,688,642 each describe a method for preparing a solid phase DNA probe array using an inkjet process. A method for detecting a target material using a solid phase probe array prepared by these methods roughly comprises (1) preparation of a solid probe array, (2) reaction process of the probe of the solid phase probe array and a target material and (3) observation after the reaction.

A general detection method will be described below using a DNA probe array as an example.

(1) Preparation of Probe Array Substrate

In the step of preparing a solid phase probe array, a DNA probe capable of specifically binding to a target material to be examined is prepared, and the DNA probe is attached on the surface of a pretreated solid phase (e.g. substrate, etc.) to form an array of spots. FIG. 1 schematically illustrates DNA probes immobilized on each spot of the solid phase probe array prepared in this step.

FIG. 1 schematically illustrates an array arrangement of spots of DNA probes fixed on a solid substrate. The figure shows an example in which DNA spots b each including the DNA probe c are spaced uniformly on a substrate of glass, plastic or the like to form a matrix. FIG. 2 is a schematic diagram showing an enlarged view of one of DNA spots in the array shown in FIG. 1. The figure schematically shows a situation of the DNA probe fixed on the substrate in each DNA spot.

(2) Treatment of Probe Array and Target Material for Detection

The treatment process of the probe of the probe array and a target material is the hybridization process in which a target material to be examined is made to contact the probe of the solid phase probe array to form a specific binding therewith. The hybridization reaction normally proceeds under a circumstance where the probe is made to contact a solution containing the target material or immersed therein at a predetermined temperature. This temperature depends on the combination of the probe and the target material.

(3) Observation After Detection (Hybridization) Process

In the observation process after the detection process, whether the target material reacts with the probe to form specific binding or not is determined using a known method, e.g. a fluorescent detection process using a fluorescence label. That is, if the target material is previously labeled with a fluorescent molecules or the like in the step of the the hybridization described in (2), only the target material bound to the solid phase probe emits fluorescence.

### SUMMARY OF THE INVENTION

Thus, if there exists a target material bound to a probe array substrate, its fluorescence is observed, which ensures that the detection process has been carried out. However, there is no way to easily determine whether or not the detection process in (2) has been carried out prior to the observation.

That is, the solid phase probe array itself does not significantly change in appearance at a first glance before and after the detection process is carried out, thus providing no absolute measure for determining whether or not the detection process has been carried out prior to the observation step. If there exists no target material to be detected in a test sample, no fluorescence is observed, making it impossible to determine whether the detection process has been actually carried out or. not even in the observation step.

Furthermore, since there is no significant change in appearance at a first glance before and after the detection process, the probe array already subjected to the detection process may be erroneously subjected to another detection process.

If the solid phase probe array is subjected to the detection process again, the amount of target material specifically bound to the probe array increases, resulting in erroneous evaluation of the concentration of target material contained in the specimen.

The present invention has been made for solving the above problems, and its object is to provide a method capable of easily determining whether or not the probe-array substrate has been already used for detecting the target material prior to the observation step, and a probe-array substrate comprising a discrimination element for use in such determination.

As a result of intensive studies for solving the above problems, the inventor has found that in the target-detection process using the probe-array substrate, the hybridization reaction usually proceeds under a circumstance where the probe is made to contact a solution containing the target material or immersed therein, and is heated to keep the probe at a predetermined temperature, and therefore if the probe-array substrate is provided with a discrimination element that changes color and/or shape when it undergoes a temperature change associated with this step, or it contacts the solution containing the target material or is immersed therein, for example, a visible change occurs in the discrimination element when the detection process is carried out. Based on the finding, the inventor has prepared the discrimination element and ensured that it can be actually used for determination, thus achieving the present invention.

Thus, one aspect of the present invention is a probe array substrate for use in a detection process of a target material comprising a probe, a substrate and a discrimination element, wherein said probe is a probe molecule capable of specifically binding to the target material, and is immobilized on a specific site on a surface of the substrate, and the discrimination element provided on the substrate shows a visible change as a detection process is carried out, whereby whether or not the probe array substrate has been subjected to the detection process is determined.

Preferably, the above probe array substrate is to be subjected to a detection process where the probe array is contacted to the test sample solution and heated for forming specific binding, and the discrimination element visibly changes with contact with liquid or with heat.

According to one embodiment of the probe array substrate of the present invention, the discrimination element changes color or shape with the above detection process.

More preferably, the visual change is a color change due to temperature change. Alternatively, the visual change may be a change in color or shape due to contact with water or a solvent contained in the test sample. More preferably, the visual change is an irreversible change. It is also preferable that the visual change of the discrimination element occurs only when the element is exposed to heat or liquid for a predetermined period of time.

The present invention also provides a probe carrier for use in detection process of a target material, which comprises a probe capable of binding to a target material fixed on said carrier; and a layer of visually recognizable water soluble composition provided on said carrier.

The present invention also provides a method for determining whether or not a probe-array substrate for use in detection process of a target material has been used for carrying out detection process of the target material using a probe array as described above, and detecting the visual change of the discrimination element.

According to the present invention, in the process for detecting a target material using a probe-array substrate, the hybridization reaction usually proceeds under a circumstance where the probe is made to contact a solution containing the target material or immersed therein, and is heated to keep the probe at a predetermined temperature, and therefore if the probe-array substrate is provided with a discrimination element changing its color and shape when it undergoes a change in temperature associated with this step, or it contacts the solution containing the target material or is immersed therein, for example, a visible change is achieved in the discrimination element when the detection process is carried out, thus making it possible to easily determine whether or not the probe-array substrate has been subjected to detection process for the target material by using the visible change as an indicator. As a result, the possibility that the probe-array substrate already used for detection process of the target material is erroneously subjected to the detection process again is eliminated, and thus process can reliably proceed to the next observation step.

### BRIEF DESCRIPTION OF THE DRAWINGS

FIG. 1 schematically shows an example of an arrayed arrangement of DNA spots on a solid phase probe array substrate;

FIG. 2 is an enlarged view schematically showing a situation in which a DNA probe is fixed in the DNA spot on the solid phase probe array substrate;

FIGS. 3A, 3B and 3C schematically show examples of a mounting form of a discrimination element in the solid phase probe array substrate comprising the discrimination element according to the present invention;

FIGS. 4A and 4B schematically show examples of a mounting form of the discrimination element using a material changing its color due to a change in temperature in the solid phase probe array substrate comprising the discrimination element according to the present invention;

FIG. 5 schematically illustrates a change in color shown by the discrimination element before and after detection process as it is subjected to a heating step, in the discrimination element using a material changing its color due to a change in temperature, which is mounted on the solid phase probe array;

FIGS. 6A and 6B schematically show a change in color occurring before and after detection process satisfying predetermined detection process conditions (FIG. 6A) and a situation in which no change in color occurs as predetermined detection process conditions are not satisfied (FIG. 6B), in the discrimination element using a material changing its color when exposed to a temperature equal to or greater than a fixed temperature for a fixed amount of time or longer, which is mounted on the solid phase probe array;

FIG. 7 schematically illustrates a change in color shown by the discrimination element before and after detection process as it contacts a specimen solution, in the discrimination element using a material changing its color or shape when wetted by water or the like, which is mounted on the solid phase probe array;

FIGS. 8A and 8B schematically show a change in color and shape occurring before and after detection process satisfying predetermined detection process conditions (FIG. 8A) and a situation in which a change in color and a change in shape are not achieved as predetermined detection process conditions are not satisfied (FIG. 8B), in the discrimination element using a material changing its color or shape when contacting water or the like for a fixed amount of time or longer, which is mounted on the solid phase probe array.

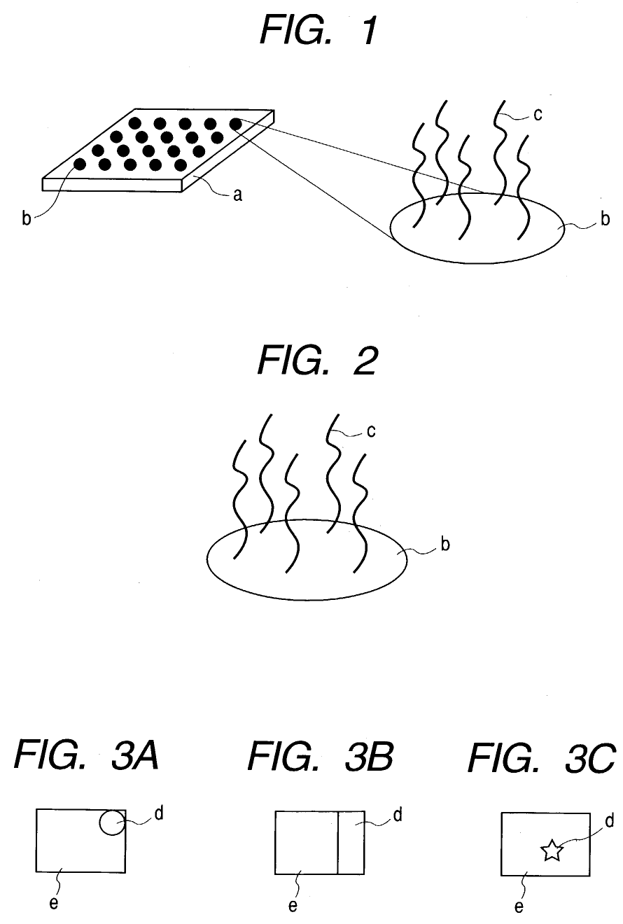

### DETAILED DESCRIPTION OF THE PREFERRED EMBODIMENTS

The present invention will be described in detail below.

In the present invention, a visual change of a discrimination element provided on a probe-array substrate is used as an indicator for determining whether or not the probe-array substrate has been subjected to detection process of a target material. Here, the visual change refers to a visually recognizable change of, for example, color or shape of the discrimination element before and after the detection process. The discrimination element is provided at a predetermined position, and observation of a certain visible change is carried out at this predetermined position, which allows easy discrimination.

Examples of discrimination elements usable as a history indicator for a probe array substrate regarding detection process in the present invention will be described below. However, the element that shows a visible change and is capable of being used is not limited thereto. And, different kinds of discrimination elements can be used for the same probe array substrate to detect different treatments.

(A) Materials usable a discrimination element in the present invention are those that change color with temperature change due to heating or cooling during detection process. More specifically, it is preferable to select a material that changes color reliably when the temperature reached a predetermined temperature for the detection process. Alternatively, one can use a material that changes color when it is heated to the reaction temperature of the detection process and then cooled. The discrimination element using a material of thermal color change may be placed on the substrate surface where probes are immobilized. Alternatively, it may be placed at a site other than the probe surface so long as it will be exposed to a predetermined temperature change during the detection process.

(B) The above-described discrimination element may be in a shape of seal, ink or sheet. Examples of the discrimination element placed on the probe array substrate are shown in FIGS. 3A-3C. Reference character e denotes a substrate of a probe array, and reference character d denotes a discrimination element. The element is preferably placed, for example, at an end or in a corner separated from the probe array area as shown in FIGS. 3A-3C, but may also be placed near the probe array region as long as it causes no problems in the detection process. The shape of the discrimination element can be selected various forms as long as visual identification is possible. Moreover, the discrimination element may be placed in the probe array area if the material has substantially no influence on the hybridization reaction between the probe and the test sample during detection process. For example, one can use a detection element being a visually recognizable water soluble composition that is partially dissolved and removed from the probe array substrate in contact with a reaction solution so that it will not affect the hybridization reaction. In this case, the surface of the probe array substrate including the probe array area may be entirely covered with the discrimination element. Alternatively,. the area of discrimination element and the DNA probe array area may be clearly separated.

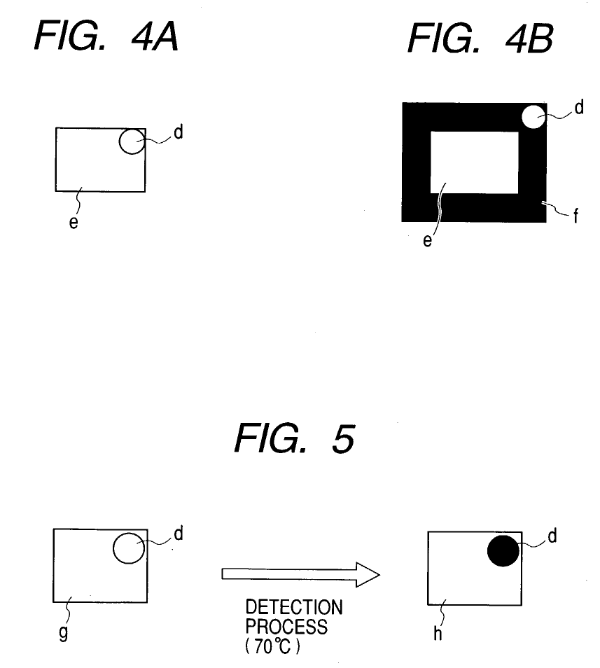

(C) The discrimination element of thermal color change is usually attached directly on the surface of the probe array substrate. Furthermore, for easy handling, the probe-array substrate may be provided with an outer periphery portion f (package portion) integrally as shown in FIG. 4B. If such an outer periphery portion (package portion) is subjected to the same heating process in the detection process, the discrimination element may be provided on this outer periphery portion (package portion) that will be exposed to the temperature change as shown in FIG. 4B.

(D) In the case where the discrimination element is mounted directly on the probe array substrate, the discrimination element may be wetted when the test sample solution is applied to the probe array during the detection process. Thus., it is desirable to use a water-resistant or solvent-resistant material for the discrimination element, or to coat the element with a water resistant or solvent resistant coating after attaching the element to the substrate, not to affect the color change. When the discrimination element is mounted outside the probe array region, the element can be placed not to be wetted, so that use of a water-resistant or solvent-resistant material is not necessary. However, the discrimination element may be wetted with water or a solvent during handling of the probe array substrate. Thus, it is preferable for the discrimination element have water and solvent resistance.

(E) If the heating temperature in the detection process is set to 70° C. for example, the discrimination element that changes the color when the temperature reaches 70° C. is attached to the probe array substrate as shown in FIG. 5. The reference character g denotes a probe array substrate before detection process, and the reference character h denotes the probe array substrate after the detection process. When the probe-array substrate is subjected to the detection process at the predetermined temperature, the color of the detection element changes, for example, from transparent to black. By visually observing and checking the change in color of the discrimination element, one can easily know that the detection process has been carried out on the probe-array substrate.

(F) When a material that changes color depending to temperature is used, it is appropriate to select a material of temperature characteristics according to the temperature conditions of the detection process. Alternatively, if only whether the probe-array substrate has been heated is determined, irrespective of the reaction temperature, one can use a material that changes color above a certain temperature.

(G) Furthermore, use of a material that changes color only when it is exposed to a predetermined or higher temperature for a predetermined or longer period of time enables control of both the temperature and period of the detection process at the same time.

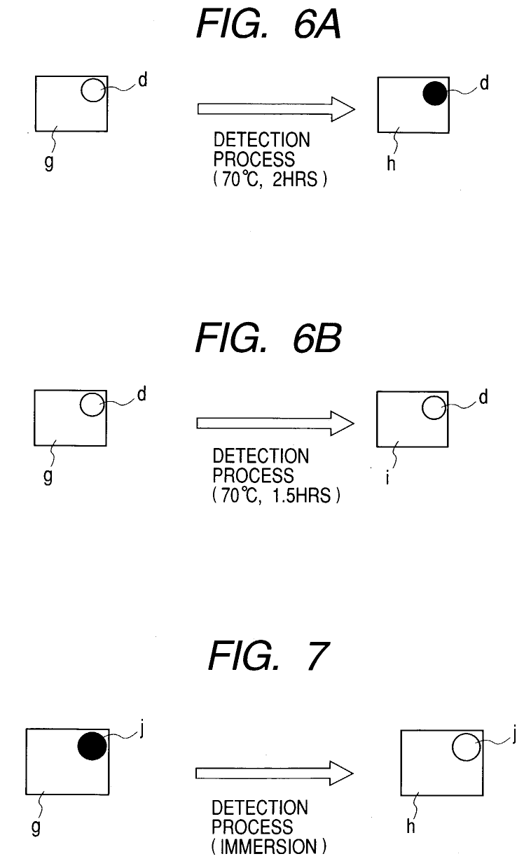

As shown schematically shown in FIGS. 6A-6B, when the conditions of the detection process are set such as 70° C. for 2 hours, the discrimination element changes color only when the reaction was carried out under the predetermined conditions (FIG. 6A), but the color will not change, for example, if the reaction time is less than 2 hours (FIG. 6B). Thus, one can easily detect whether or not the predetermined conditions were satisfied based on this difference. Here, reference character i denotes a probe array subjected to insufficient detection process.

(H) Preferably, this visible change is irreversible. For example, discrimination elements that changes color irreversibly can be prepared by using rewritable materials based on amorphous-crystal or phase separation-non-phase separation described below.

(a) Electron-Donating Coloring Compounds

There are electron-donating organic compounds such as leuko auramines, diarylphthalides polyarylcarbinols, acyl auramines, rhodamine B lactams, indolines, spiropyrans, fluorans, cyanine pigments and crystal violet.

(b) Electron-Accepting Compounds

There are oxides such as phenols, phenol metallic salts, metallic carbonates, sulfonic acid, sulfonates, phosphates, metallic phosphates, acidic phosphates, acidic metallic phosphates, phosphates, phosphorous acids, metallic phosphates, etc.

(c) Reversibility Agents

Reversibility agents are materials that can change interaction between the electron-donating coloring compound and the electron-accepting compound indirectly and reversibly enhancing or inhibiting the interaction. Examples of such reversibility agents are cholesterol, pregnenolone and steroids.

This type of compound becomes colorless instantly when it is heated to the melting point temperature or above by a thermal head and then rapidly cooled down. Meanwhile it gradually develops color when it is heated to or above the glass transition point of the ink. The temperature and the time required for color development can be controlled by the concentration of the reversibility agent to be added.

(I) It is also possible to use a material that changes color and/or shape when in contact with a solvent of a test sample in a solution or liquid form for the discrimination element. When such a material is used, the probe array substrate may be marked with a mark that looses color when immersed in or contacted with water etc. Here the mark is used as a discrimination element. Such a simple and convenient method may also be employed because the purpose can be fully achieved by confirming the disappearance of the mark after the detection process due to the contact with the solvent contained in the test sample.

The discrimination element using a material that changes color or shape only when it contacts the solvent or the like contained in the test sample may have any position and shape as long as it is placed on the probe array substrate as shown in FIGS. 3A-3C. However, the discrimination element should be contacted, or immersed in water together with the probe array, so that it is preferable that the discrimination element is mounted directly on the substrate portion of the probe array substrate rather than the package portion.

(J) When the discrimination element j using the material that changes color or shape when wetted with water etc., for example, the element is black before detection process, but turns white after detection process where it contacts the sample solution and is wetted with water etc. By visually observing and checking the change in color in the discrimination element after detection process, whether the detection process has been carried out or not can easily be determined.

(K) Furthermore, use of a material that changes color and/or shape only when it is exposed to water etc. for a predetermined or longer period of time enables control of the period of the detection process at the same time.

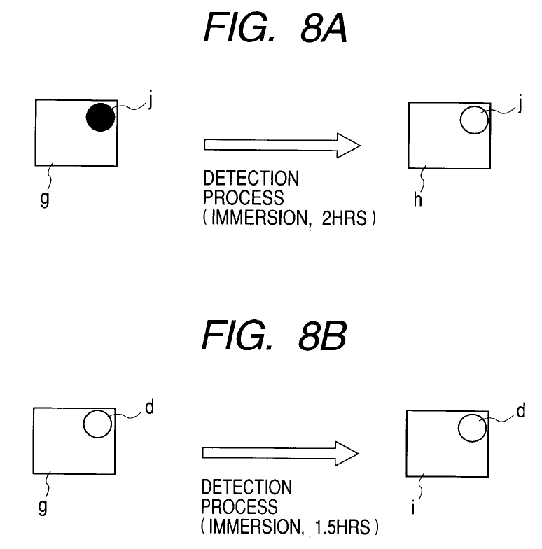

As shown schematically shown in FIGS. 8A-8B, when the conditions of the detection process are set such as 70° C. for 2 hours, the discrimination element changes color only when it has contacted water for the predetermined reaction time (FIG. 8A), but the color will not change, for example, if the reaction time is less than 2 hours (FIG. 8B). Thus, one can easily detect whether or not the predetermined conditions were satisfied based on this difference.

(L) The discrimination element using a material that changes color and/or shape when wetted with water etc. can be prepared by providing a water-soluble colored layer on the substrate, applying directly to the substrate a water soluble colored resin prepared by dissolving or dispersing dye or pigment as a colorant in a water soluble resin. This colored layer may be provided on a base sheet to form a discrimination element in a label form. Such a label is attached to the probe array substrate with an adhesive or the like, and the color will change or fade when the label is immersed in the test sample solution, thus making it possible to determine whether it has contacted the solution. Alternatively, one can use a coloring composition which comprises at least one electron-accepting compound and electron-donating compound and discolors in contact with water to detect whether or not the element has contacted water by observing discoloring of the label.

### EXAMPLES

The present invention will be described more in detail below showing Examples. The Examples each represents one example of the best mode according to the present invention, but the present invention is not limited to the embodiments of these Examples.

### Example 1

Probe array substrate provided with discrimination element using a material that changes color according to temperature, and process for detecting a target material using the probe array substrate.

(1) Preparation of DNA Probe Array

A DNA probe array is prepared according to the process described in Japanese Patent Laid-Open No. 11-187900.

First, a glass substrate is subjected to ultrasonic cleaning and alkali cleaning, and then subjected to a surface treatment with a silane coupling agent containing a silane compound having an amino group bonded thereto. Then the amino group is reacted with N-maleimidecaproiloxysuccinimide (EMCS) so that the amino groups originating from the silane coupling agent react with the succinimide groups to introduce maleimide groups derived from EMCS to the substrate surface.

Separately, a DNA oligomer having a base sequence of 5′ACTGGCCGTCGTTTTACA3′(SEQ ID NO:1) with the 5′ terminal modified with a sulfanile group is synthesized as a single-stranded DNA probe, and a solution containing this single-stranded DNA probe is spotted on the surface-treated glass substrate using an inkjet process. As a result, the maleimide group reacts with the sulfanile group at the 5′ terminus of the probe to fix the probe, whereby a DNA probe is prepared.

(2) Determination of whether the substrate has been subjected to a hybridization treatment or not using seal-type discrimination element

The prepared DNA probe array was dipped in a 2% bovine serum albumin aqueous solution and left standing for 2 hours for blocking the substrate surface. Onto the back surface of the DNA probe array, is attached a seal-type discrimination element containing a material that changes color at 45° C. (Commercial name: Thermolabel 3E, Nichiyu Giken Kogyo Co., Ltd.).

Using the DNA probe array with the discrimination element attached thereto, the detection process was carried out at 45° C. for 2 hours in an aqueous solution containing a target material. In this case, the target material was a single-stranded DNA (5′TGTAAAACGACGGCCAGT3′) (SEQ ID NO:2) complementary to the DNA probe of (1). As a result, the material used for the discrimination element being white in color before the hybridization process, changed its color to black after the hybridization process.

According to the color change of the discrimination element, it was easy to know that the detection process had been carried out or not. (3) Result of observation after detection process

In addition, fluorescence observation was made using a scanner for DNA microarray (trade name: GenePix 4000B; manufactured by Axon Instruments, Inc.) on the DNA probe array after the detection process. The fluorescent level was 8008.5.

Thus, whether the substrate has been subjected to the detection process or not can easily be determined by visual inspection prior to the fluorescent observation without affecting florescent observation, according to the discrimination element prepared by using a material that changes color depending to the temperature.

### Example 2

Probe array substrate provided with a discrimination element utilizing a material that changes color when wetted with water, and detection process of target material using this probe array substrate.

(1) Preparation of DNA Probe Array

The DNA probe array was prepared in the same manner as in (1) of Example 1.

(2) Determination of Whether the Aubstrate was Subjected to the Detection Process by Using Marker-Type Discrimination Element

The prepared DNA probe array was dipped in a 2% bovine serum albumin aqueous solution and left standing for 2 hours for blocking the substrate surface. Onto the back surface of the substrate, letters were written by using a material that changes color when wetted with water as a marker type discrimination element (Commercial name: Fushigi Pen, a product of Econet).

Using the DNA probe array provided with the discrimination element thereto, the detection (hybridization) process was carried out at 45° C. for 2 hours in an aqueous solution containing a target material, where the target material was a single-stranded DNA (5′TGTAAAACGACGGCCAGT3′)(SEQ ID NO:2) having a base sequence complementary to the DNA probe described in the item (1) of Example 2. As a result, the material used for the discrimination element that had been black before the detection process, became colorless and transparent after the detection process.

According to the color change of the discrimination element, it was easy to know that the detection process had been carried out. (3) Result of observation after detection process

In addition, fluorescence observation was made using a scanner for DNA microarray (trade name: GenePix 4000B; manufactured by Axon Instruments, Inc.) on the DNA probe array after the detection process. The fluorescent level was 4749.4.

Thus, whether the substrate has been subjected to the detection process or not can easily be determined by visual inspection prior to the fluorescent observation without affecting florescent observation, according to the discrimination element prepared by using a material that changes color when wetted with water.

### Example 3

Probe array substrate provided with a discrimination element utilizing a material that changes color and shape when wetted with water, and detection process of target material using this probe array substrate.

(1) Preparation of DNA Probe Array

The DNA probe array was prepared in the same manner as in (1) of Example 1.

(2) Determination of Whether the Substrate was Subjected to the Detection Process by Using Sheet-Type Discrimination Element

The prepared DNA probe array was dipped in a 2% bovine serum albumin aqueous solution and left standing for 2 hours for blocking the substrate surface. Onto the back surface of the substrate, a sheet-type discrimination element utilizing a material that changes color and shape when wetted with water was attached (Commercial name: Aqualabel, a product of Tokyo Deodorant).

Using the DNA probe array provided with the discrimination element thereto, the detection (hybridization) process was carried out at 45° C. for 2 hours in an aqueous solution containing a target material, where the target material was a single-stranded DNA (5′TGTAAAACGACGGCCAGT3′)(SEQ ID NO:2) having a base sequence complementary to the DNA probe described in the item (1) of Example 3. As a result, the material used for the discrimination element that had been white before the detection process, became translucent and peeled off from the substrate at the end of the detection process.

According to the change in color and shape of the discrimination element, it was easy to know that the detection process had been carried out.

(3) Result of Observation after Detection Process

In addition, fluorescence observation was made using a scanner for DNA microarray (trade name: GenePix 4000B; manufactured by Axon Instruments, Inc.) on the DNA probe array after the detection process. The fluorescent level was 4722.

Thus, whether the substrate has been subjected to the detection process or not can easily be determined by visual inspection prior to the fluorescent observation without affecting florescent observation, according to the discrimination element prepared by using a material that changes color and shape when wetted with water.

What is claimed is:

1. A probe array substrate for use in a detection process of a target material in a specimen comprising a probe, a substrate and a discrimination element,
wherein said probe is a probe molecule capable of specifically binding to the target material, and is immobilized on a specific site on a surface of the substrate, and
the discrimination element provided on the substrate shows a visible change as a detection process is carried out, whereby whether or not the probe array substrate has been subjected to the detection process is determined.

2. A method for determining whether or not a probe array substrate for use in detection process of a target material has been used for carrying out detection process of the target material,
wherein the probe array substrate is such that
said probe is a probe molecule capable of specifically binding to the target material, and is fixed in a specific site on a surface of a substrate, and
the probe array substrate is provided thereon with an element showing a visible change as detection process of the target material is carried out, as an element for determining whether or not the detection process of the target material has been carried out, and
whether or not said probe-array substrate has been used for detection process of the target material is determined by identifying the existence or nonexistence of a visible change shown by said discrimination element.

3. The probe array substrate according to claim 1, wherein said detection process of the target material comprises,
as a step of making the specimen contact the surface of the substrate with the probe fixed thereon to achieve said specific binding,
making a specimen in the form of an aqueous solution or liquid phase contact the surface of the substrate and subjecting the specimen to a heat treatment while the specimen contacts the surface of the substrate, and
said element is an element showing a visible change when contacting the specimen in the form of an aqueous solution or liquid phase, or subjected to the heat treatment while contacting the specimen.

4. The method according to claim 2, wherein said detection process of the target material comprises,
as a step of making the specimen contact the surface of the substrate with the probe fixed thereon to achieve said specific binding, making a specimen in the form of an aqueous solution or liquid phase contact the surface of the substrate and subjecting the specimen to a heat treatment while the specimen contacts the surface of the substrate,
said element is an element showing a visible change when contacting the specimen in the form of an aqueous solution or liquid phase, or subjected to the heat treatment while contacting the specimen, and
whether or not said probe-array substrate has been used for detection process of the target material is determined by identifying the existence or nonexistence of a visible change shown by said discrimination element.

5. The probe array substrate according to claim 1, wherein said change shown by the element includes a change in color or change in shape.

6. The probe array substrate according to claim 1, wherein said discrimination element uses a material changing its color due to a change in temperature.

7. The probe array substrate according to claim 1, wherein said discrimination element uses a material changing its color or shape when contacting a solvent contained in the specimen in the form of an aqueous solution or liquid phase.

8. The method according to claim 4, wherein said visible change is an irreversible change.

9. The method according to claim 4, wherein said discrimination element is placed in a site on the surface of the substrate allowing the discrimination element to contact the specimen or undergo a change in temperature by the heat treatment, at the time when the element is made to contact said specimen in the form of an aqueous solution or liquid phase or subjected to the heat treatment while contacting the specimen.

10. The probe array substrate according to claim 1, wherein in said discrimination element, the size of an area showing a visible change is set to a visually identifiable size.

11. The probe array substrate according to claim 1, wherein said visible change is achieved by using a material changing its color when exposed to a temperature equal to or higher than a predetermined temperature for a fixed amount of time or longer.

12. The probe array substrate according to claim 1, wherein said visible change is achieved by using a material changing its color and shape when contacting a solvent in the specimen in the form of an aqueous solution or liquid phase for a fixed amount of time or longer.

13. The probe array substrate according to claim 1, wherein the probe array comprises more than one discrimination element, each being an indicator of different treatment conditions.

14. A probe carrier for use in detection process of a target material, comprising:
A) a probe capable of binding to a target material fixed on said carrier; and
B) a layer of visually recognizable water soluble composition provided on said carrier.


SEQUENCE LISTING (2 sequences)

SEQ ID NO: 1 - DNA, 18 bp, artificial
  probe
actggccgtcgttttaca

SEQ ID NO: 2 - DNA, 18 bp, artificial
  target
tgtaaaacgacggccagt